(19) United States
(12) Patent Application Publication
(10) Pub. No.: US 2012/0040033 A1
(43) Pub. Date: Feb. 16, 2012

(54) Anti-infective mangrove extract against viruses, bacteria and fungi

(76) Inventor: Ernest Timothy Armstrong, Palm Desert, CA (US)

(21) Appl. No.: 13/136,806

(22) Filed: Aug. 10, 2011

Related U.S. Application Data
(60) Provisional application No. 61/401,213, filed on Aug. 10, 2010.

(51) Int. Cl. A61K 36/185; A61K 36/85; A61K 38/12; A61K 38/08; A61K 38/15; A61P 31/04; A61K 31/421; A61K 31/715; A61K 31/70; A61P 35/00; A61P 31/12; A61P 31/10; A61K 36/19; A61K 31/425

(52) U.S. Cl. 424/769; 514/3.7; 514/2.9; 514/3.6; 514/2.4; 514/3.3; 514/19.2; 514/359; 514/374; 514/54; 514/23

(57) ABSTRACT

The current invention discloses drug formulations of anti-infective mangrove extracts and their ability to block the reproductive capabilities of viruses, funguses, bacteria and tumors. Mangrove extracts can be ingested internally to prevent the proliferation of viruses, funguses, bacteria and tumors in humans and other animals; or the mangrove extracts can be applied directly to the skin, in the form of a disinfectant hand gel, to fight microbial infection.

### CROSS REFERENCE TO RELATED APPLICATION

This application claims the priority right from the U.S. provisional patent application 61/401,213 that was filed on Aug. 10, 2010, the content of which is herewith incorporated in its entirety by reference.

### BACKGROUND OF THE INVENTION

The current invention discloses drug formulations of anti-infective mangrove extracts and their ability to block the reproductive capabilities of viruses, funguses, bacteria and tumors. Mangrove extracts can be ingested internally to prevent the proliferation of viruses, funguses, bacteria and tumors in humans and other animals; or the mangrove extracts can be applied directly to the skin, in the form of a disinfectant, sanitizing hand gel, to fight microbial infection.

The growth and reproduction of viruses, funguses, bacteria and tumors depends on cellular reproduction which in turn is dependant on the splitting and replication of the DNA double helix. For the DNA to cleave, a complex (known as a DNA topoisomerase) must act to introduce a break. It is the discovery of the inventor of the current invention that certain biological compounds found in mangrove trees can block the cleavage complexes, thereby blocking DNA replication and cellular reproduction.

Mangrove trees are one of the few trees that can survive partially submerged in marine environments. Mangrove trees, living in warm, shallow marine waters must produce substances needed to fight off those marine viruses, funguses and bacteria; microbial organisms which are not generally encountered by trees in the terrestrial world.

All species that inhabit the outer (ocean-facing) portion of the mangrove swamp are halophytes, i.e., plants that are adapted to saline soils, and certain mangrove species can tolerate soils more than double the salinity of ocean water. Species that are less resistant to salt damage grow on the landward edge of mangal, where high tides reach only infrequently, or along river banks (estuarine mangrove), where freshwater mingles with sea water due to tidal influences.

It is well documented that several marine animals, which interestingly enough live on or near mangrove rhizomes, produce defensive biochemical compounds broadly known as secondary metabolites with antiproliferative properties against viruses, funguses and bacteria. Several of these defensive compounds are considered to have the potential to become pharmacological agents. Although such medically important compounds have been discovered in those marine animals, including sea squirts (Tunicata, Ascidiacea), sponges, soft corals and mollusks; harvesting those marine animals in quantities sufficient for commercial medical applications has proven not to be practical or economic.

It was the discovery by the inventor of the current patent that certain extracts obtained from the rhizomes and other parts of the mangrove tree also contain compounds with anti-infective properties. This discovery is of great importance because large scale harvesting of mangroves is practical, economic, not particularly environmentally damaging and because the extraction of the active substances from the rhizomes is relatively easy.

Mangrove rhizome extract described in the current invention can be used by humans, animals or plants to prevent and treat a variety of diseases, with the essential being those diseases which depend on cellular division and replication.

Following are descriptions of original clinical studies using a mangrove extract on humans diagnosed with the common cold virus conducted by immunologists at the Atlanta Allergy and Asthma Clinic, Atlanta, Ga:

Example Clinical Study 1.) Methods: A water-based treatment was produced from dried, crushed Rhizophora mangle (Stylosa, Selala and Samonensis) rhizome and placed in liquid 1 ml droppers. Sixty (n=60) male and female patients, aged 2 to 80, having common cold symptoms were administered 2 droppers 4 times a day for 48 hours. Patients self-rated their symptoms and documented their concomitant cold medication use on a diary card. Results: Over 80 percent of the 60 patients reported “complete resolution” of their cold symptoms within 48 hours.

Example Clinical Study 2.) Methods: From February 2004 through February 2005, male and female human subjects between the ages of 3 and 74 years with demonstrated infection to the common cold were enrolled for study participation. As an inclusion criterion, subjects needed to present with two or more symptoms consistent with having been infected with viral-induced common cold: (i.e. nasal congestion, runny nose, sore throat, headache, cough with or without chest congestion and malaise). All subjects received active treatments containing a concentrated mangrove extract from the rhizomes of Rhizophora Mangle (Stylosa, Selala and Samonensis). Rhizophora mangle is recognized by the United States Department of Agriculture as an approved dietary supplement. There were two forms of delivery: approximately 50% of the subjects received a study medication with extract instilled in a water-based treatment while the 50% received extract in a glycerin-based treatment. The water-based treatment was produced from 50 g of dried, crushed Rhizophora rhizome in 1.9 liters of water. The extraction was obtained with a hot water decoction, which produced a dark brown, thick liquid, the UV spectrum at 280 nm, Abs. 0.2972. The yield was 29 ml/100 g from crushed, dried Rhizophora. The final concentration was 3.4 g/ml (d˜1.2) or 2 ml/0.25 liter of water. This fluid extract included a preservative, sodium benzoate. The delivery system was a 1 oz. brown actinic bottle with a 1 ml capacity dropper. The water-based treatment was made by Terradyne Naturale, Inc. in their laboratories in Woodbine, Iowa, USA. The glycerin-based treatment was manufactured by a serialized technology process designed to produce a dark brown, high viscosity liquid, with a specific gravity of 1.190, and a standardized brix density of 53 (+/−2%). The yield is from crushed, dried Rhizophora. The glycerin-based extract used no preservative, was alcohol-free, was stored in and delivered with a 1 ml capacity dropper. The glycerin-based treatment was made by Cedar Bear Naturales, Inc. in their laboratories in Roosevelt, Utah, USA. Subjects were administered one or the other of the study medications over the course of 48 hours. All subjects were instructed to self-dose as follows: Place droppers of extract drawn from a 30 ml (1 fluid oz) brown actinic bottle into a cup containing 0.025-liter of water; and drink 4 times daily (morning, noon, evening and at bedtime) for two consecutive days. Subjects receiving the water-based treatment used two (2) full 1 ml droppers each dosing, while subjects receiving the glycerin-based treatment used one (1) full 1 ml dropper each dosing. Self-rated symptom diaries in the form of a standardized clinical report were maintained by the subjects to document symptom severity. Interviews with the subjects were used to monitor protocol adherence. Results: Of the 84 subjects from whom a clinical report was obtained, 79% reported substantial to complete resolution of their cold symptoms within 12 to 48 hours of the first use of the study medication. The remaining 21% of the subjects reported less than substantial to no improvement. Of significant note is that several of the patients with recurrent sinusitis were in the 79% group. Based on prior experience of the investigator, it appears that the use of antibiotics by several of the patients with chronic sinusitis was less than historical observations. The glycerin-based treatment group on average reported smaller doses of their study medication than did the water-based treatment group. No adverse events were reported.

A list of viral infections includes, but is not limited to the following viruses and conditions; the Common Cold Virus (Coronaviruses, Rhinoviruses, and others), Herpes Simplex Virus (types I and II), Hepatitis, Influenza, Parainfluenza, Dengue Fever, Human Papillomavirus (HPV), Avian Flu (such as strain H5N1), Human Immunodeficiency Virus (HIV) and the like.

A list of such fungal infections includes, but is not limited to the following funguses and conditions; Aspergillosis, Blastomycosis, Cocci (Coccidioidomycosis), Cryptococcosis, Fungal Infections of Fingernails and Toenails, Fungal Pneumonia, Fungal Sinusitis, Histoplasmosis, Hypersensitivity Pneumonitis, Mucormycosis, Nail Fungus, Paracoccidioidomycosis, Sporotrichosis, Valley Fever and the like.

A list of such bacterial infections includes, but is not limited to the following bacteria and conditions; Nontuberculous Mycobacteria (NTM), Brucellosis, Campylobacter, Cholera, Glanders, Hansen's Disease, Helicobacter pylori, Hot Tub Lung, Pneumococcal Diseases, Leprosy, Leptospirosis, Melioidosis, Q Fever, Septic Arthritis, Shigellosis, Tularemia, Typhoid Fever, Vibrio, Parahaemolyticus, Vibrio Vulnificus, Yersinia Enterocolitica and the like.

A list of such tumors includes, but is not limited to the following conditions; cancer cells in animals or humans bearing a neoplastic disease, for example, Myelocystic Leukemia, Lymphocystic Leukemia, Melanoma, Adenocarcinoma of the lung, Neuroblastoma, Carcinoma of the Lung, Breast Carcinoma, Colon Carcinoma, Bladder Carcinoma, Endometrial Cancer, Nasopharyngeal Cancer, Osteosarcoma, Prostate Cancer, Testicular Cancer, Vulvar Cancer, Stomach Cancer and the like.

The types of mangroves of particular interest to the current invention include, but are not limited to Rhizophora Mangle or Red Mangrove (Rhizophora (Selala, Stylosa or Samoensis); Black Mangrove (Avicenniaceae, Acanthaceae, Verbenaceae, avicennia germinans)); and White Mangrove (Combretaceae (Conocarpus, Laguncularia or Lumnitzera)).

Mangrove rhizome extract described in the current invention can be delivered to humans to prevent and treat infections by many different routes of delivery. A list of such delivery vehicles includes, but is not limited to; liquid, drops, dots, elixirs, solutions, suspensions, powders, film strips, creams, ointments, tablets, lozenges, capsules, injections and towels made of paper, cloth and the like. Each of said delivery vehicles can have excipients, binding agents, penetration enhancers and other inactive ingredients. Other delivery means are possible to one skilled in the art.

In one preferred embodiment of the delivery, the current invention can be administered to humans as a liquid or gel in which said human has liquid or gel dispersed via a dispenser onto the hands. Such anti-microbial liquid or gel dispensers are commonly found outside of stores and other public places with a variety of anti-microbial and anti-bacterial ingredients in them. The current invention discloses formulations which include mangrove extracts in the anti-microbial and anti-bacterial liquids or gels.

Mangrove rhizome extract described in the current invention can be administered to humans or animals in need of preventing or treating infections in high or low doses, with the essential being that the dose is a disease-opposing amount for viral, fungal or bacterial infections. For example, a concentration of between 0.1% and 77% mangrove extract is contained in the chosen delivery vehicle.

Mangrove extract can be used to prevent and treat infections as a prophylactic treatment prior to infection or as a preventative medication before and during an infection, or as a treatment after infection has occurred.

Mangrove rhizome extract can be used to prevent and treat infections by itself or in combination with other antiviral, antifungal or antibacterial agents.

An embodiment of particular interest to the current patent includes an extract containing cyclic peptides (including but not limited to ulicylamide, ulithiacyclamide, the heptapeptide lissoclinamides, the octapeptide patellamides, didemnin B, a cyclic depsipeptide and bicyclic depsipeptide). Compounds containing thiazole and oxazoline amino acids are a preferred embodiment. Other compounds of interest to the current patent are CNS membrane-active toxins, ion channel effectors and polysaccharides that interact with DNA and micro-filament processes. Compounds of particular interest to the current patent are ecteinascidin compounds (especially ecteinascidin 743), which may interfere in nucleotide excision repair, a process in which damaged bases are fixed.

As a drug substance, mangrove extract can be utilized in its native form, or chemically altered into related chemical entities and derivatives such as metabolites, acids, hydroxyl groups, esters, salts, alcohols, acyl groups, and so forth in ways which would be obvious to one skilled in the art.

### MANUFACTURING AND PREPARATION

For the current invention, the whole plant or parts thereof such as cortices, bark, flowers, seeds, roots, rhizomes, and stems or combinations thereof may be used. One preferred embodiment of the current invention is the bark from the rhizomes.

The condensed plant product(s) is used in the raw, in the dried state, or in the form crushed or pulverized after drying. It is preferable to use fabricated products crushed or pulverized after drying.

### PRIOR ART

R. mucronata is used as an astringent and to treat angina, haemorrhaging (extracts from the seedlings in Indochina); diarrhoea (China, Japan): diabetes, dysentery, hematuria. A poultice of the leaves are used to relief armoured fish stings. Old leaves and roots are used during childbirth (Malay). Bark is used to treat blood in the urine (Burma).

Hematuria (blood in the urine) has many different causes, and can come from an infection (including viral), inflammation, or injury to the urinary system.

According to Garcia-Barriga (1975) Kino de Colombia, resin from the red mangrove, has several medicinal uses. One Cali doctor reports a cure of throat cancer, with gargles of mangrove bark (Garcia-Barriga, 1975). Reported to be astringent, emmenagogue, expectorant, hemostat, styptic, and tonic, red mangrove is a folk remedy for angina, asthma, backache, boils, ciguatera, convulsions, diarrhea, dysentery, dyspepsia, elephantiasis, enuresis, epistaxis, eye ailments, fever, filariasis, hemoptysis, hemorrhage, inflammation, jaundice, leprosy, lesions, leucorrhea, malignancies, scrofula, short wind, sores, sorethroat, syphilis, tuberculosis, uterorrhagia, and wounds (Duke and Wain, 1981; Morton, 1981).

Rhizophora mangrove L. is reported among the American plants killing fungi. Sánchez, 1998, outlines that the healing properties conferred to this plant are due to their composition in tannins and some compounds.

The essential volatile and compound oils, or semi-volatiles oils that are also present in Rhizophora mangrove L. watery bark extract are used for antimicrobial purposes either against gram-positive and gram-negative bacteria or mushrooms (Hussein, 1990) (Mount and col., 1991) (Made and Irobi, 1993) (Chinou and col., 1996).

It was demonstrated that Rhizophora mangrove L (trade name Cikrón V) is able to stimulate healing wounds in rabbits, Figueroa and col. (1995), and to prevent the umbilical infections in calves (Figueredo and cabbage., 1995).

According to a study by Figueroa and col. (1995), the solution was not found to cause irritability, but to act as a protecting film that accelerates healing in a 100% of the cases studied, causing no inflammation either and diminishing the number of ulcers.

Mangrove is a folk remedy for angina, asthma, boils, diarrhea, dysentery, eye ailments, fever, hemorrhage, inflammation, jaundice, leprosy, sores, sorethroat, and wounds.

Other significant evaluations were carried out by (Garcia and col., 1998) (Bulnes and col., 2000). By means of the test of irritability, carried out after having used Rhizophora mangrove extract, no hystopathological alterations were observed.

Studies carried out with Rhizophora mangrove L watery and alcoholic roots and shafts extracts demonstrate their inhibitory activity against both Gram positive and Gram negative bacteria of human interest. (Red and Limit, 1978).

The potential value of mangrove bark tannins as cytotoxic and/or antineoplastic agents and as antimicrobial agents has been demonstrated.

Mangrove plants are rich sources of saponins, alkaloids and flavonoids. Plant saponins have been shown to have interesting biological activities such as spermicidal and molluscicidal activity.

The extraction of natural chemical compounds, in addition to those already known to the pharmacopoeia of the people, continues to this day and among the latest additions are an array of substances from glues to alkaloids and saponins and many other substances of interest to modern industry and medicine.

U.S. Pat. No. 4,631,149 refers to marine organisms.

U.S. Pat. No. 5,135,746 refers to diseases caused by protozoan parasites.

U.S. Pat. No. 5,256,663 refers to marine organisms.

U.S. Pat. No. 5,929,047 refers to an anti-oxidant activity and a free radical-scavenging activity, while the current patent refers to blocking cellular reproduction through blocking DNA replication. Additionally U.S. Pat. No. 5,929,047 refers to a different type of plant, which is also commonly called “mangrove”, Momordica charantia L., or Aspalathus linearis belonging to the family Leguminosae. According to National Geographic (February 2007, p. 136) “There are some 70 species [of mangrove] from two dozen families—among them palm, hibiscus, holly, plumbago, acanthus, legumes, and myrtle.” The current invention relates only to mangroves of the family Rhizophoraceae.

U.S. Pat. No. 6,544,560 refers to marine organisms.

U.S. Pat. No. 6,777,004 refers to a different type of plant, which is also commonly called “mangrove”, the Aegiceras corniculatum (Blanco) belonging to the family Myrsinaceae.

### REFERENCES

Agriculture Handbook 165. 1960. Index of plant diseases in the United States. USGPO. Washington.

Cannell, M. G. R. 1982. World forest biomass and primary production data. Academic Press, New York.

Duke, J. A. 1972. Isthmian ethnobotanical dictionary. Publ. by the author. Harrod & Co., Baltimore.

Duke, J. A. 1981b. The gene revolution. Paper 1. p. 89-150. In: Office of Technology Assessment, Background papers for innovative biological technologies for lesser developed countries. USGPO. Washington.

Duke, J. A. and Atchley, A. A. 1984. Proximate analysis. In: Christie, B. R. (ed.), The handbook of plant science in agriculture. CRC Press, Inc., Boca Raton, Fla.

Duke, J. A. and Wain, K. K. 1981. Medicinal plants of the world. Computer index with more than 85,000 entries. 3 vols.

Irvine, F. R. 1961. Woody plants of Ghana. Oxford University Press, London.

Little, E. L. Jr. 1983. Common fuelwood crops: a handbook for their identification.

McClain Morton, J. F. 1965. Can the red mangrove provide food, feed, and fertilizer. Econ. Bot. 19:113-123.

Morton, J. F. 1981. Atlas of medicinal plants of middle America. Bahamas to Yucatan. C. C. Thomas, Springfield, Ill.

Murry, M. A. and Benemann, J. R. 1981. Freshwater plants. p. 407-470. In: McClure, T. A. and Lipinsky, E. S. (eds.), CRC handbook of biosolar resources. vol. II. Resource materials. CRC Press, Inc., Boca Raton, Fla.

N. A. S. 1980a. Firewood crops. Shrub and tree species for energy production. National Academy of Sciences, Washington, D.C.

Michael Mastaller, “Mangroves: The Forgotten Forest Between Land and Sea”, Tropical Press, 1997 (p. 28-30: about the roots; p. 34-35: about how seedlings disperse and sprout; p. 97: medicinal uses).

What is claimed is:

1. A composition for inhibiting the cellular reproduction of viruses, funguses, bacteria and tumors wherein said composition comprises administering to a human or animal in need thereof a disease-opposing amount of mangrove extract.

2. A composition according to claim 1. wherein said mangrove is selected from Rhizophora (Selala, Stylosa or Samoensis); Avicenniaceae, Acanthaceae, Verbenaceae (Avicennia germinans); or Combretaceae (Conocarpus, Laguncularia or Lumnitzera).

3. A composition according to claim 1. wherein said extract is selected from the mangrove's cortices, bark, flowers, seeds, roots, prop roots, rhizomes or stems; or combinations thereof.

4. A pharmaceutical composition according to claim 1. wherein said extract is a pharmaceutically-acceptable carrier and an antiviral, antifungal, antibacterial or antitumor compound wherein said compound is selected from the group consisting of cyclic peptides such as ulicylamide, ulithiacyclamide, the heptapeptide lissoclinamides, the octapeptide patellamides, didemnin B, a cyclic depsipeptide and bicyclic depsipeptide), thiazole, oxazoline amino acids, polysaccharides, ecteinascidin compounds, tannins and/or glycosides; or their respective metabolites, acids, hydroxyl groups, esters, salts, alcohols and acyl groups thereof.

5. A composition according to claim 1. wherein said viruses, funguses, bacteria and tumors are selected from the following: Coronaviruses, Rhinoviruses, Herpes Simplex Virus (types I and II), Hepatitis, Influenza, Parainfluenza, Dengue Fever, Human Papillomavirus, Avian Flu, Avian Flu strain H5N1, and Human Immunodeficiency Virus; Aspergillosis, Blastomycosis, Cocci (Coccidioidomycosis), Cryptococcosis, Fungal Infections of Fingernails and Toenails, Fungal Pneumonia, Fungal Sinusitis, Histoplasmosis, Hypersensitivity Pneumonitis, Mucormycosis, Nail Fungus, Paracoccidioidomycosis, Sporotrichosis, and Valley Fever; Nontuberculous Mycobacteria, Brucellosis, Campylobacter, Cholera, Glanders, Hansen's Disease, Helicobacter pylori, Pneumococca, Leprosy, Leptospirosis, Melioidosis, Q Fever, Septic Arthritis, Shigellosis, Tularemia, Typhoid Fever, Vibrio, Parahaemolyticus, Vibrio Vulnificus, Yersinia and Enterocolitica; or Myelocystic Leukemia, Lymphocystic Leukemia, Melanoma, Adenocarcinoma of the lung, Neuroblastoma, Carcinoma of the Lung, Breast Carcinoma, Colon Carcinoma, Bladder Carcinoma, Endometrial Cancer, Melanoma, Colorectal Cancer, Pancreatic Cancer, Nasopharyngeal Cancer, Osteosarcoma, Prostate Cancer, Testicular Cancer, Vulvar Cancer, and Stomach Cancer.

6. A composition according to claim 1. wherein said administration is delivered to as a liquid, lotion, cream or gel via a dispenser onto the hands and other regions of the skin.

7. A composition according to claim 1. wherein said administration is given to a human or animal in need thereof once, twice or thrice daily.

8. A composition according to claim 1. wherein said administration is given to a human or animal in need thereof between 1 and 42 times.

9. A composition according to claim 1. wherein said administration is given to a human or animal in need thereof in a concentration of between 1 percent extract to 99 percent excipient to 99 percent extract to 1 percent excipient.

10. A composition according to claim 1. wherein the delivery of said administration to a human or animal in need thereof is selected from liquids, drops, dots, elixirs, solutions, suspensions, powders, tablets, capsules, lozenges, film strips, syrups, creams, injections, ointments, gels and infused on towels.

11. A composition according to claim 1. wherein said disease-opposing amount is given to a human or animal in need thereof in a dose of between 0.001 mL and 1000 mL.

12. A method for inhibiting the cellular reproduction of viruses, funguses, bacteria and tumors wherein said method comprises administering to a human or animal in need thereof a disease-opposing amount of mangrove extract.

13. A method according to claim 12. wherein said mangrove is selected from Rhizophora (Selala, Stylosa or Samoensis); Avicenniaceae, Acanthaceae, Verbenaceae (Avicennia germinans); or Combretaceae (Conocarpus, Laguncularia or Lumnitzera).

14. A method according to claim 12. wherein said extract is selected from the mangrove's cortices, bark, flowers, seeds, roots, prop roots, rhizomes or stems; or combinations thereof.

15. A method according to claim 12. wherein said extract is a pharmaceutically-acceptable carrier and an antiviral, antifungal, antibacterial or antitumor compound wherein said compound is selected from the group consisting of cyclic peptides such as ulicylamide, ulithiacyclamide, the heptapeptide lissoclinamides, the octapeptide patellamides, didemnin B, a cyclic depsipeptide and bicyclic depsipeptide), thiazole, oxazoline amino acids, polysaccharides, ecteinascidin compounds, tannins and/or glycosides; or their respective metabolites, acids, hydroxyl groups, esters, salts, alcohols and acyl groups thereof.

16. A method according to claim 12. wherein said viruses, funguses, bacteria and tumors are selected from the following: Coronaviruses, Rhinoviruses, Herpes Simplex Virus (types I and II), Hepatitis, Influenza, Parainfluenza, Dengue Fever, Human Papillomavirus, Avian Flu, Avian Flu strain H5N1, and Human Immunodeficiency Virus; Aspergillosis, Blastomycosis, Cocci (Coccidioidomycosis), Cryptococcosis, Fungal Infections of Fingernails and Toenails, Fungal Pneumonia, Fungal Sinusitis, Histoplasmosis, Hypersensitivity Pneumonitis, Mucormycosis, Nail Fungus, Paracoccidioidomycosis, Sporotrichosis, and Valley Fever; Nontuberculous Mycobacteria, Brucellosis, Campylobacter, Cholera, Glanders, Hansen's Disease, Helicobacter pylori, Pneumococca, Leprosy, Leptospirosis, Melioidosis, Q Fever, Septic Arthritis, Shigellosis, Tularemia, Typhoid Fever, Vibrio, Parahaemolyticus, Vibrio Vulnificus, Yersinia and Enterocolitica; or Myelocystic Leukemia, Lymphocystic Leukemia, Melanoma, Adenocarcinoma of the lung, Neuroblastoma, Carcinoma of the Lung, Breast Carcinoma, Colon Carcinoma, Bladder Carcinoma, Endometrial Cancer, Melanoma, Colorectal Cancer, Pancreatic Cancer, Nasopharyngeal Cancer, Osteosarcoma, Prostate Cancer, Testicular Cancer, Vulvar Cancer, and Stomach Cancer.

17. A method according to claim 12. wherein said administration is delivered to as a liquid, lotion, cream or gel via a dispenser onto the hands and other regions of the skin.

18. A method according to claim 12. wherein said administration is given to a human or animal in need thereof once, twice or thrice daily in a concentration of between 1 percent extract to 99 percent excipient to 99 percent extract to 1 percent excipient.

19. A method according to claim 12. wherein the delivery of said administration to a human or animal in need thereof is selected from liquids, drops, dots, elixirs, solutions, suspensions, powders, tablets, capsules, lozenges, film strips, syrups, creams, injections, ointments, gels and infused on towels.

20. A method according to claim 12. wherein said disease-opposing amount is given to a human or animal in need thereof in a dose of between 0.001 mL and 1000 mL.
